(19) United States
(12) Patent Application Publication
(10) Pub. No.: US 2024/0389707 A1
(43) Pub. Date: Nov. 28, 2024

(54) INSOLE AND FOOT PRESSURE MEASURING ARRANGEMENT

(71) Applicant: Benecke-Kaliko AG, Hannover (DE)

(72) Inventors: Sima Farazi, Hannover (DE); Felix Lindner, Isernhagen (DE); Gabriel Cristian Hidalgo Lucio, Hannover (DE); Nsombo Nlandi, Würselen (DE)

(73) Assignee: Benecke-Kaliko AG, Hannover (DE)

(21) Appl. No.: 18/696,545

(22) PCT Filed: Aug. 15, 2022
(86) PCT No.: PCT/DE2022/200182

§ 371 Date: Mar. 28, 2024

(30) Foreign Application Priority Data
Sep. 28, 2021 (DE) 10 2021 210 800.8

(51) Int. Cl. A43B 3/44; A43B 17/00; A61B 5/103

(52) U.S. Cl. CPC A43B 3/44; A43B 17/00; A61B 5/1036; A61B 2503/10

(57) ABSTRACT

The invention relates to an insole, in particular for a sports shoe, having: a flexible flat substrate which is geometrically adapted to the inner contour of the sports shoe and a pressure sensor arrangement which is fastened to the substrate and consists of a plurality of flat pressure sensors, each having a pair of power supply and sensor signal lines for supplying power to the pressure sensors and passing on the sensor signals to an integrated or external sensor signal transmission unit (15; 15′), wherein 2 to 4 pressure sensors are provided in the toe region of the insole, one of which pressure sensors is placed in the region of the big toe, 2 to 4 pressure sensors are provided in the ball region, 1 to 2 pressure sensors are provided in the region of the outside of the foot, and 2 to 4 pressure sensors are provided in the heel region, and wherein the active area of each sensor is 2 cm² or more.

### BRIEF SUMMARY

The invention relates to an insole, in particular for a sports shoe, having: a flexible flat substrate which is geometrically adapted to the inner contour of the sports shoe and a pressure sensor arrangement which is fastened to the substrate and consists of a plurality of flat pressure sensors, each having a pair of power supply and sensor signal lines for supplying power to the pressure sensors and passing on the sensor signals to an integrated or external sensor signal transmission unit. It furthermore relates to a foot pressure measurement arrangement which comprises such an insole, and finally to a sports shoe.

In order to improve performance, it has long been known in certain sports to carry out an analysis of the foot movements of the sportsperson and in particular of the pressure exerted on the ground by different parts of the sole of the foot. Such analyses were already employed in golf a long time ago, wherein the golfer was placed on a sufficiently large pressure measurement plate which uses a multiplicity of sensor points arranged in a matrix to enable a highly spatially resolved measurement of the pressure exerted on the ground by the individual regions of the foot when hitting the golf ball. It goes without saying that such an arrangement having a relatively large and heavy measuring sensor is primarily suitable for stationary use and its possible applications are therefore limited.

Furthermore, it has long been known to integrate sensors in the sole of shoes, in particular also sports shoes, for the purpose of measuring certain physiological parameters or also the pressure exerted on the ground by the wearer during certain movements. These solutions have the disadvantage that if the sole or other parts of the shoe construction are unusable due to wear or damage, the relatively costly sensor system becomes unusable too. In addition, the disposal of the shoe then requires separate disposal of the electronics, which of course increases the disposal costs. Moreover, this results in relatively high total costs for sportspeople who use several pairs of shoes for training purposes, if each pair of these shoes is equipped with a corresponding sensor system.

Meanwhile, solutions were therefore also proposed in which sensors for physiological parameters or pressure or force sensors are not integrated in the sole of the shoe but in an insole which can be used by various shoes and does not become worthless when a shoe is worn out.

An insole of this type for medical purposes is known from US 2010/0324455 A1, and a system for measuring the force expended by the feet during cycling which includes a corresponding insole is known from US 20015/0025816 A1. Furthermore, US 2019/0041278 A1 teaches a force measuring device which in particular can be built into an insole for a shoe.

An insole.

The invention is based on the object of providing an improved insole of the generic type which delivers reliable and easy-to-evaluate data, in particular even when it is mass-produced with a substantially identical configuration and in a few different sizes, and is used for a wide range of sportspeople and possibly also for various sports.

Expedient configurations of an insole are provided. Furthermore, foot pressure measurement arrangements are provided.

The invention is based on experimental analyses by the inventor, specifically in the domain of golf, and includes the idea of providing the insole in each case with a suitable number of pressure sensors in the toe region, in the ball region, in the region of the outside of the foot and in the heel region, and of designing these sensors to have an active area that is large enough that reliable and easy-to-evaluate measurement results can be obtained despite certain anatomical differences in different wearers of the sole and namely without time-consuming prior calibrations for the respective wearer.

Specifically, it is proposed that 2 to 4 pressure sensors are provided in the toe region of the insole, one of which pressure sensors is placed in the region of the big toe, 2 to 4 pressure sensors are provided in the ball region, 1 to 2 pressure sensors are provided in the region of the outside of the foot, and 2 to 4 pressure sensors are provided in the heel region, and wherein the active area of each sensor is 2 cm² or more.

The total number of sensors here is small enough for simple transmission and evaluation but allows meaningful pressure signals to be obtained, in particular for different sports, such as, for instance, golf, tennis, baseball or running sports.

In expedient configurations, the active area of the pressure sensors is in the range between 2.5 cm² and 5 cm².

In another practically meaningful configuration, provision is made for all the pressure sensors to be substantially rectangular in shape and to have the same geometric shape and active area. This allows inexpensive production of the insole and of insoles which are intended for different shoe sizes.

In one particular implementation, two pressure sensors are arranged in the toe region, one of which pressure sensors extends over the contact area of at least two toes. The particularly relevant pressure forces which are exerted on the ground, on the one hand, by the big toe and, on the other hand, by the middle toes of the foot can thus be measured sufficiently accurately and then undergo simple evaluation.

In another implementation, provision is made for three pressure sensors to be arranged in the ball region (that is to say in that region of the sole of the foot which adjoins the toe region dorsally), one of which pressure sensors is situated medially, another laterally and the third centrally between the two.

In another expedient implementation, one pressure sensor is arranged in the region of the outside of the foot substantially centrally over the longitudinal extent of the outside of the foot. This individual sensor, during normal use for the above-mentioned sports, allows sufficiently reliable measurement of the pressure forces occurring in this region.

Finally, in another implementation, provision is made for three pressure sensors to be arranged in the heel region, one of which pressure sensors is placed medially, one laterally and one dorsally. For this implementation, the same as for the above-mentioned configurations applies that fully sufficiently resolved pressure force measurement during the relevant movements of the sportsperson is possible, namely using only a few sensors.

In another implementation which is expedient according to the findings of the inventor, the substantially rectangularly shaped pressure sensors (possibly rounded at the corners) are positioned in such a way that the longitudinal directions form angles of between 0° and 30° with the direction of the longitudinal extent of the insole.

In one implementation of the pressure sensors which is preferred from the current perspective, the pressure sensors are resistive dielectric pressure sensors, which can also be referred to as pressure measurement film sensors. In one specific implementation, these pressure sensors comprise a first conductive layer, a dielectric layer on the latter, which dielectric layer is surrounded and delimited by a spacer that determines the shape of the pressure sensor, and a second conductive layer on the dielectric layer and the spacer. This layer sequence can be arranged, on the one hand, directly on the flat flexible substrate of the insole or else, on the other hand, on a specific thin isolating sensor substrate which includes one or more pressure sensors having the respectively associated supply line structure.

In a proven implementation which works well from the current perspective, provision is made for the first conductive layer to comprise silver, for the dielectric layer to be formed of dielectric islands spaced apart from one another, and for the second conductive layer to comprise carbon.

Alternatively, the pressure sensors can be formed according to other physical active principles, specifically be capacitative or piezoresistive sensors so long as they allow a low overall height to be achieved, allowing them to be easily integrated in an insole.

In one expedient structural implementation, the insole has an at least 3-layer structure which, in addition to the flat flexible substrate, comprises an intermediate layer, in which the pressure sensors and the power supply and sensor signal lines are arranged, and one or more top layers, wherein the rigidity of the flat substrate is greater than the rigidity of the one or more top layers. Specifically, the flat flexible substrate and the top layer or at least one of the top layers here can be based on PET.

The proposed insole can be implemented in a minimal configuration in which it contains no power supply components, signal transmission components or evaluation components and is therefore able to be produced particularly easily and inexpensively. A foot pressure measurement arrangement formed with such an insole comprises, in addition to the actual insole, a strip-shaped supply line substrate which has a first end formed on the insole (or on the above-mentioned separate pressure sensor substrate) or connected therewith and a second end in the form of a connection socket or connection plug or the like. Furthermore, it comprises a power supply and transmission module which is able to be connected to the second end of the supply line substrate and which has at least one power source for supplying power to the pressure sensors and a sensor signal transmission unit for transmitting the sensor signals to the outside and is configured to be attached to the sports shoe. The sensor signal transmission unit can in particular be a Bluetooth transmission unit.

Such a foot pressure measurement arrangement can be equipped with an integrated processing unit which is incorporated in the sensor signal lines between the pressure sensors and the wireless transmission unit and which in particular comprises a number of pre-processing channels that corresponds to the number of pressure sensors in order to pre-process the sensor signals of the individual pressure sensors separately. Such an integrated processing unit can in particular perform digitalization and/or other pre-processing of the raw sensor signals for the transmission to the outside.

### BRIEF DESCRIPTION OF THE SEVERAL VIEWS OF THE DRAWING(S)

Advantages and expediencies of the invention furthermore emerge from the following description of exemplary embodiments on the basis of the figures. In the figures:

FIG. 1 shows a plan view of an insole according to the invention (without top layer(s)),

FIGS. 2A-2F show schematic illustrations of the sensor configuration of the insole according to FIG. 1 (in each case with a corresponding detail view), illustrating various phases of the production of the pressure sensors,

FIG. 3 shows a schematic illustration of a first implementation of the foot pressure measurement arrangement according to the invention and

FIG. 4 shows a schematic illustration of a second implementation of the foot pressure measurement arrangement according to the invention.

### DETAILED DESCRIPTION

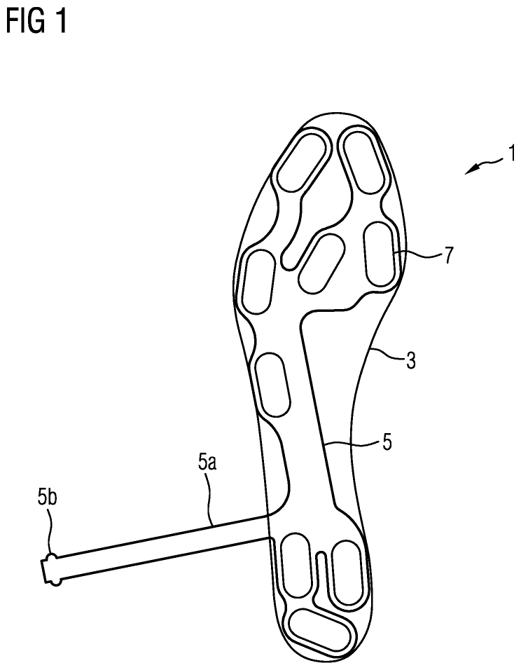

FIG. 1 shows, as essential parts of an insole 1, a foot-shaped PET substrate 3 and a sensor substrate film 5 fastened thereto, on which sensor substrate film are arranged a total of nine pressure sensors 7 each having a pair of power supply and sensor signal lines (not shown here). The sensor substrate film 5 has a strip-shaped lateral extension 5a, which can be led upward to the upper of a sports shoe in which the insole 1 is laid, and has a connection plug 5b at its end.

Of the nine pressure sensors, two are provided in the toe region, one of which pressure sensors is placed in the region of the big toe and the other of which is placed such that it extends over the contact area of two other toes. Three further pressure sensors are arranged in the region dorsal to the toe region equidistantly from medial to lateral, one further pressure sensor is placed approximately centrally in the region of the outside of a foot that is stepping on the insole, and the three remaining pressure sensors are placed in the heel region, namely one medially, one laterally and one dorsally.

The pressure sensors 7 can have a width in the range between 12 and 18 mm, in particular of about 15 mm, and—in different tested implementations—a length in the range between 15 mm and 40 mm, in particular between 30 and 35 mm. As can be seen in FIG. 1, their basic shape is that of a rounded rectangle and, in the implementation shown, all the pressure sensors have the same shape and—with the exception of the dorsal pressure sensor in the heel region—are oriented substantially parallel to the longitudinal direction of the insole 1.

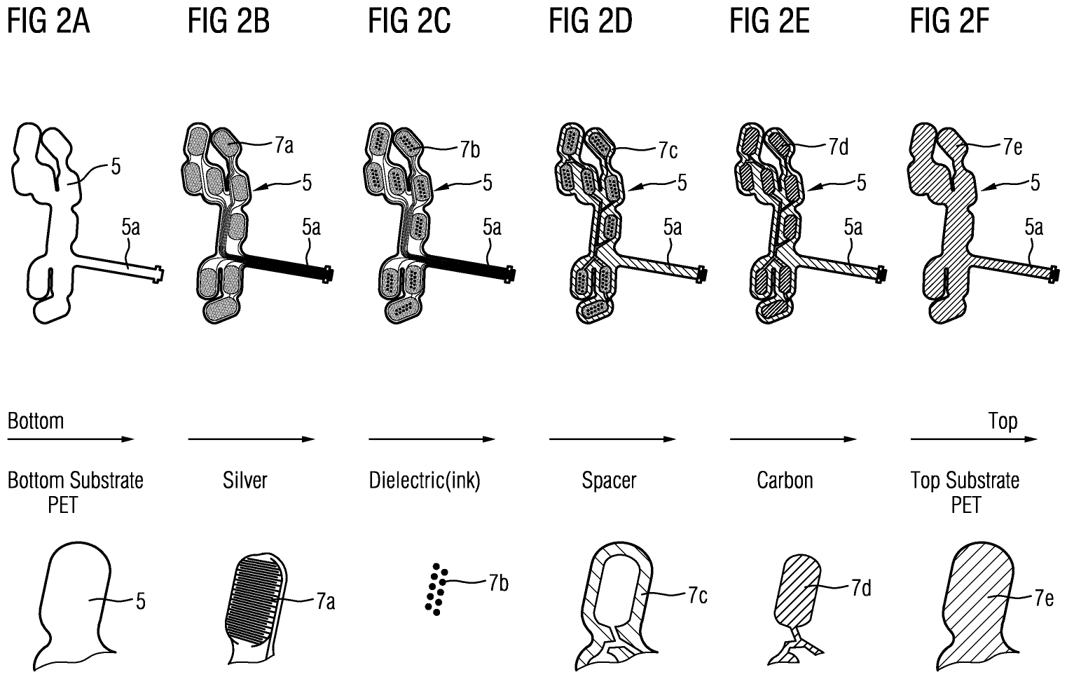

FIGS. 2A to 2F schematically show the essential phases of the production of the pressure sensor system of the insole in an implementation which is expedient from the current perspective. Accordingly, in a first step (FIG. 2A), the sensor substrate film 5 is provided in a suitable geometric shape. In a second step (FIG. 2B), a silver layer 7a is deposited in each of the area regions provided for the individual pressure sensors. In a third step (FIG. 2C), a dielectric layer 7b composed of small disk-shaped dielectric aggregates is applied in the area regions of the silver layer of the individual sensors, for example by means of a screen-printing process or inkjet printing or the like.

In a fourth step (FIG. 2D), an insulating spacer 7c, which delimits the active area of the sensors, is applied around the dielectric in each case. In a fifth step (FIG. 2E), a carbon layer 7d is applied to the dielectric 7b and the spacer 7c as a second conductive layer of the sensor. In a final, sixth step (FIG. 2F), the sensor substrate film with the sensor components produced thereon is covered and sealed by a PET top film 7e.

It goes without saying that a first supply line is formed together with the first conductive layer, the silver layer 7a, and a second conductive supply line is formed together with the second conductive layer, the carbon layer 7d, said supply lines leading to the respective pressure sensor for supplying power thereto and for sensor signal derivation. This is not illustrated separately in FIGS. 2B and 2E.

The pressure sensor configuration produced in the way described above is laminated between the pre-produced insole substrate 3 and at least one top layer, wherein methods known per se can be used. In principle, it is also possible to produce the first conductive layer of the pressure sensors directly on an insole substrate and to form the top layer of the sensor configuration at the same time as top layer of the entire insole.

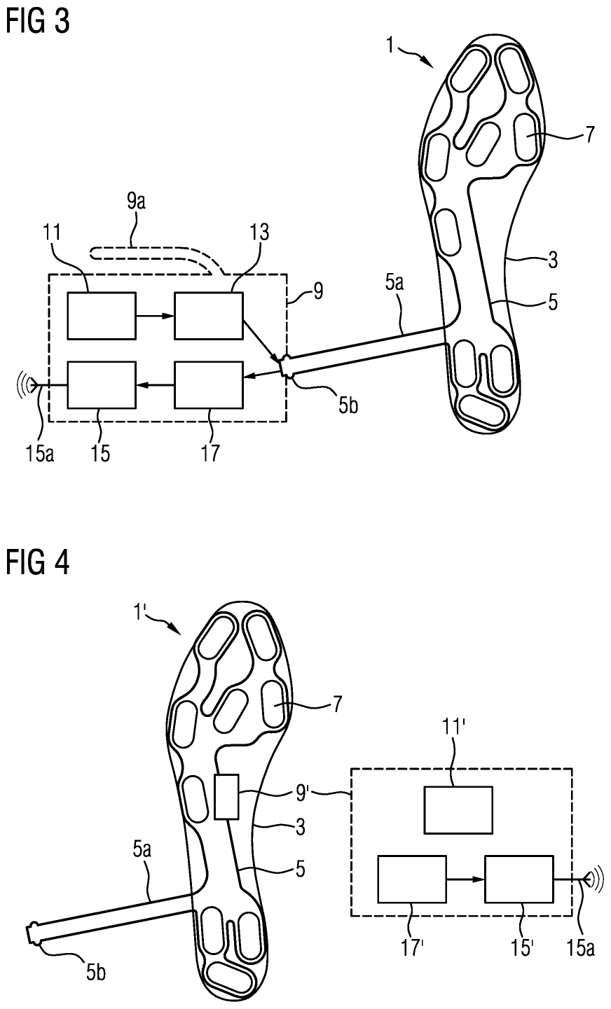

FIG. 3 schematically shows, in the form of a block diagram, a foot pressure measurement arrangement comprising the insole 1 according to FIG. 1 and, in addition to the insole, comprising a power supply and transmission module 9. This module is able to be attached, by means of a schematically illustrated clip 9a, to the sports shoe in which the insole 1 is laid.

The module 9 is connected to the connection 5b of the insole 1 and comprises a power source 11, for instance a rechargeable battery, a power supply controller 13, which is connected between the rechargeable battery and the supply lines to the pressure sensors 7, which supply lines are combined in the plug 5b, in order to supply power to the pressure sensors. It further comprises a wireless transmission unit 15, for instance of Bluetooth type, which transmits the pressure data obtained by the insole 1, via a corresponding antenna 15a, to outside the sports shoe for evaluation. In the implementation shown, a processing unit 17 is connected between the signal connection 5b of the insole 1 and the transmission unit 15, which processing unit performs pre-processing of the sensor signals for the wireless signal transmission.

The power supply and transmission module 9 interacts with an evaluation system in respect of evaluating the signals from the pressure sensors, which evaluation system can be implemented for example in the smartphone of a user of the sports shoe, but this does not belong to the present invention and is therefore not described here.

FIG. 4 shows another implementation of the foot pressure measurement arrangement, in which essential electronics components are integrated in the insole 1. It goes without saying that for this purpose the corresponding supply lines to the sensors are not led out of the sole via a strip-shaped supply line substrate but are connected to the corresponding components within the sole.

In the implementation shown, the foot pressure measurement arrangement comprises a power supply element 11′ integrated in the insole, as well as a Bluetooth transmission unit 15′ likewise integrated in the insole 1 and a processing unit 17′, the functions of the latter two units substantially corresponding to the functions of the above-described corresponding units 15 and 17 from FIG. 3.

### LIST OF REFERENCE SIGNS

1; 1′ insole
3 substrate
5 sensor substrate film
5a extension
5b connection plug
7 pressure sensors
7a silver layer
7b dielectric layer
7c spacer
7d carbon layer
7e top film
9; 9′ transmission module
9a clip
11, 11′ power source/power supply element
13 power supply controller
15, 15′ wireless transmission unit
17, 17′ processing unit

1-15. (canceled)

16. An insole for a sports shoe, comprising:
a flexible flat substrate, which is geometrically adapted to the inner contour of the sports shoe; and
a pressure sensor arrangement which is fastened to the substrate and consists of a plurality of flat pressure sensors, each having a pair of power supply and sensor signal lines for supplying power to the pressure sensors and passing on the sensor signals to an integrated or external sensor signal transmission unit;
wherein 2 to 4 pressure sensors of the plurality of flat pressure sensors are provided in the toe region of the insole, one of which pressure sensors is placed in the region of the big toe, 2 to 4 pressure sensors are provided in the ball region, 1 to 2 pressure sensors are provided in the region of the outside of the foot, and 2 to 4 pressure sensors are provided in the heel region, and wherein the active area of each sensor is 2 cm2 or more.

17. The insole of claim 16, wherein the active area of the plurality of pressure sensors is in the range between 2.5 cm2 and 5 cm2.

18. The insole of claim 16, wherein the plurality of pressure sensors are rectangular in shape and have the same geometric shape and active area.

19. The insole of claim 16, wherein two pressure sensors are arranged in the toe region, one of which pressure sensors extends over the contact area of at least two toes, and;
wherein three pressure sensors are arranged in the ball region, one of which pressure sensors is situated medially, another laterally and the third centrally between the two;
wherein one pressure sensor is arranged in the region of the outside of the foot substantially centrally over the longitudinal extent of the outside of the foot; and
wherein three pressure sensors are arranged in the heel region, one of which pressure sensors is placed medially, one laterally and one dorsally.

20. The insole of claim 16, wherein longitudinal directions of the plurality of pressure sensors have angles of between 0° and 30° with the direction of the longitudinal extent of the insole.

21. The insole of claim 16, wherein the plurality of pressure sensors are resistive dielectric pressure sensors, in particular pressure measurement film sensors.

22. The insole of claim 21, wherein the plurality of pressure sensors comprise a first conductive layer, a dielectric layer on the first conductive layer, which the dielectric layer is surrounded and delimited by a spacer that determines the shape of the pressure sensor, and a second conductive layer on the dielectric layer and the spacer.

23. The insole of claim 22, wherein the first conductive layer comprises silver, the dielectric layer is formed of dielectric islands spaced apart from one another, and the second conductive layer comprises carbon.

24. The insole of claim 16, wherein the plurality of pressure sensors are capacitive or piezoresistive sensors.

25. The insole of claim 16, wherein the insole further comprising an at least 3-layer structure which, in addition to the flat flexible substrate, comprises an intermediate layer, in which the pressure sensors and the power supply and sensor signal lines are arranged, and one or more top layers, wherein the rigidity of the flat substrate is greater than the rigidity of the one or more top layers.

26. A foot pressure measurement arrangement comprising:
an insole having a plurality of pressure sensors;
a strip-shaped supply line substrate, which has a first end formed on the insole or connected to the insole and a second end in the form of a connection socket, and a power supply and transmission module which is able to be connected to the second end of the supply line substrate and which has at least one power source for supplying power to the pressure sensors and a sensor signal transmission unit for transmitting the sensor signals to the outside and is configured to be attached to the sports shoe.

27. A foot pressure measurement arrangement comprising:
an insole having a plurality of pressure sensors;
a power source for supplying power to the pressure sensors which is integrated in the insole, and an integrated wireless transmission unit for transmitting the sensor signals to the outside which is connected to the free ends of the sensor signal lines.

28. The foot pressure measurement arrangement of claim 27, wherein the integrated power source is of the piezoelectric type and configured in such a way that it converts movements of the foot of the wearer into electrical energy, or is of the thermoelectric type and configured in such a way that it converts the body heat of the wearer into electrical current, or is in the form of a film solid-state battery or film solid-state rechargeable battery.

29. The foot pressure measurement arrangement of claim 27, having an integrated processing unit (17; 17′) which is incorporated in the sensor signal lines between the pressure sensors (7) and the wireless transmission unit and which comprises a number of pre-processing channels that corresponds to the number of pressure sensors in order to pre-process the sensor signals of the individual pressure sensors separately.

30. The foot pressure arrangement of claim 27, the arrangement incorporated in a sports shoe, in particular a golf, tennis, baseball or running shoe.
